(19) United States
(12) Patent Application Publication
(10) Pub. No.: US 2014/0100244 A1
(43) Pub. Date: Apr. 10, 2014

(54) Preventive And/Or Therapeutic Agent For Thromboembolism In Thromboembolism Patient With Severe Renal Impairment

(71) Applicant: Daiichi Sankyo Company, Limited, Tokyo (JP)

(72) Inventors: Tetsuya Kimura, Tokyo (JP); Tomohiko Kumakura, Tokyo (JP); Masaya Tachibana, Tokyo (JP); Chiaki Matsumoto, Tokyo (JP); Kenji Abe, Tokyo (JP)

(73) Assignee: Daiichi Sankyo Company, Limited, Tokyo (JP)

(21) Appl. No.: 14/041,681

(22) Filed: Sep. 30, 2013

Related U.S. Application Data
(63) Continuation-in-part of application No. 13/554,610, filed on Jul. 20, 2012, now abandoned.

(30) Foreign Application Priority Data
Dec. 14, 2011 (JP) 2011-273516

(51) Int. Cl. A61K 31/444

(52) U.S. Cl. CPC A61K 31/444
USPC 514/301

(57) ABSTRACT

It is intended to provide a highly safe, orally administrable preventive and/or therapeutic agent for thrombosis and/or embolism that can be applied to a thrombosis and/or embolism patient with severe renal impairment. The present inventors have found that even for a thrombosis and/or embolism patient with severe renal impairment, use of edoxaban at a dose of 15 mg once a day can effectively prevent thrombosis and/or embolism while avoiding the risk of bleeding. The present inventors have also found that even for a thrombosis and/or embolism patient with severe renal impairment, edoxaban at a dose of 15 mg once a day can effectively prevent thrombosis and/or embolism with safety over a long period.

This application is a continuation in part of a national application Ser. No. 13/554,610 filed on Jul. 20, 2012, which claims the benefit of Japanese Patent Application Number JP 2011-273516, filed on Dec. 14, 2011, which is hereby incorporated by reference.

### FIELD OF THE INVENTION

The present invention relates to a preventive and/or therapeutic agent for thrombosis and/or embolism in a thrombosis and/or embolism patient with severe renal impairment, containing edoxaban.

### BACKGROUND

Oral anticoagulant drugs substituting for warfarin have been remarkably developed in recent years, including the newly found direct thrombin inhibitor, dabigatran, and activated blood coagulation factor X (referred to as FXa herein) inhibitors edoxaban, rivaroxaban, and apixaban.

Edoxaban tosilate hydrate (Patent literature 1 and Patent literature 2) is sold in Japan as a tablet under the trade name of LIXIANA (registered trademark) tablet for the reduction of occurrence of venous thromboembolism (referred to as VTE herein) in patients undergoing total knee replacement (referred to as TKR herein), total hip replacement (referred to as THR herein), or hip fracture surgery (referred to as HFS herein) as an indication. The edoxaban tosilate hydrate is also under a multinational clinical trial at phase-III for cerebral infarction or systemic embolism attributed to non-valvular atrial fibrillation (referred to as NVAF herein) as an indication (Non-patent literature 1 and Non-patent literature 2).

For the typical dosage and administration of the LIXIANA (registered trademark) tablet, 30 mg of edoxaban is orally administered to an adult once a day. The LIXIANA (registered trademark) tablet may cause an elevated serum concentration of edoxaban in patients with renal functional impairment, increasing the risk of bleeding. Thus, to a patient with moderate renal impairment (creatinine clearance (referred to as CL_{CR} herein): 30 mL/min or higher but lower than 50 mL/min), the LIXIANA (registered trademark) tablet is administered at an appropriately reduced dose of 15 mg once a day after evaluation of each individual patient for his or her risk of onset of venous thromboembolism and risk of bleeding. In addition, the use of the LIXIANA (registered trademark) tablet is contraindicated to patients with severe renal impairment (CL_{CR}: lower than 30 mL/min) (Non-patent literature 3). Also, it is known that the dose of a pharmaceutical composition containing edoxaban may be selected on the basis of the reference value of a dose determinant in a patient in need of administration thereof (Patent literature 3).

Edoxaban was subjected to a clinical trial targeting individuals with renal functional impairment, in which edoxaban was studied for its pharmacokinetics in the individuals with renal functional impairment (Non-patent literature 4). In this clinical trial, 15 mg of edoxaban was administered at a single dose to each patient in five groups differing in the severity of renal functional impairment.

Assessment Reports, etc., evaluating LIXIANA (registered trademark) tablet for the reduction of occurrence of VTE in patients undergoing TKR, THR, or HFS as an indication or efficacy, describe applicant's recognition, in view of few clinical trials from patients having a CL_{CR} lower than 30 mL/min, that: the determination of whether LIXIANA (registered trademark) tablet should or should not be administered to these patients requires careful consideration of the risk of VTE compared to the risk of bleeding; and in cases where it is determined that the drug should be administered, the dose needs to be reduced. The Assessment Reports, etc. also describe the decision by the Japan Pharmaceuticals and Medical Devices Agency and the expert committee of the Expert Council that the use of LIXIANA (registered trademark) tablet should be contraindicated to patients having a CL_{CR} lower than 30 mL/min based on: the unknown safety of LIXIANA (registered trademark) tablet in these patients; the possibility that the benefit of avoiding VTE could be accompanied by the unacceptable risk of bleeding; and the risk of VTE can be reduced by an approach other than the administration of the anticoagulant drug (Non-patent literature 5 to 9).

Dabigatran, rivaroxaban, and apixaban are under clinical trial or have been approved for each thromboembolism as an indication. All of these compounds are under clinical trial with exclusion criteria set for individuals with renal functional impairment or have been approved on the condition that the use of each compound is made at an appropriately reduced dose or with caution, is not recommended, or is contraindicated to individuals with renal functional impairment on the basis of each clinical trial. Edoxaban, rivaroxaban, apixaban, and dabigatran are common in that they are low-molecular compounds acting on a blood coagulation cascade centered on FXa or thrombin, while these compounds are known to largely differ in chemical structure and also in pharmacokinetics such as metabolic pathway, excretion pathway, the rate of protein binding, bioavailability, a terminal half-life (referred to as t_{1/2} herein), and/or a time to maximum plasma concentration (tₘₐₓ) (Non-patent literature 10).

There is a demand for a highly safe, orally administrable preventive and/or therapeutic agent for thrombosis and/or embolism in a thrombosis and/or embolism patient with severe renal impairment. Particularly, atrial fibrillation (referred to as AF herein) patients with severe renal impairment are a population at a high risk of thrombosis and/or embolism in need of long-term anticoagulant therapy. Thus, there is a demand for the development of a highly safe, orally administrable anticoagulant agent that can be applied to these patients.

### CITATION

Patent literature 1: WO2003/000657
Patent literature 2: WO2003/000680
Patent literature 3: WO2010/071164
Non-patent literature 1: Thromb. Haemost. 2010 September; 104 (3): 633-41
Non-patent literature 2: Am. Heart J. 2010 October; 160 (4): 635-41
Non-patent literature 3: LIXIANA (registered trademark) tablets, package insert, the 2nd revised edition in July 2011
Non-patent literature 4: Homepage of information on ethical pharmaceuticals review (http://www.info.pmda.go.jp/approvalSrch/PharmacySrchInit ?), Pharmaceuticals and Medical Devices Agency, LIXIANA (registered trademark) tablets, The Brief Summary of Application Material, 2.7.6 Summary of Individual Studies, 114-128, Web published on Jul. 25, 2011
Non-patent literature 5: Homepage of information on ethical pharmaceuticals review (http://www.info.pmda.go.jp/approvalSrch/PharmacySrchInit ?), Pharmaceuticals and Medical Devices Agency, LIXIANA (registered trademark) tablets, The Brief Summary of Application Material, 2.5 Global Assessment for Clinical Practice 48-76, Web published on Jul. 25, 2011
Non-patent literature 6: Homepage of information on ethical pharmaceuticals review (http://www.info.pmda.go.jp/approvalSrch/PharmacySrchInit ?), Pharmaceuticals and Medical Devices Agency, LIXIANA (registered trademark) tablets, The Brief Summary of Application Material, 2.7.2 Clinical Pharmacological Study, 30-32 Section “2.3.2 PK for Renal Functional Impairment in Europe”, Web published on Jul. 25, 2011
Non-patent literature 7: Assessment Report as of Feb. 9, 2011 for LIXIANA (registered trademark) tablets, 41-43 Section “(2) Validity of Reduced Dose for Renal Functional Impairment Patient and for combined use with P-gp inhibitor”, Web published on Jul. 25, 2011
Non-patent literature 8: Assessment Report as of Feb. 9, 2011 for LIXIANA (registered trademark) tablets, 66-69 Section “(7)1 Individual with Renal Functional Impairment”, Web published on Jul. 25, 2011
Non-patent literature 9: Assessment Report as of Feb. 9, 2011 for LIXIANA (registered trademark) tablets, 74-78, Web published on Jul. 25, 2011
Non-patent literature 10: Circ. J., 2011, Vol. 75, 1539-1547

### SUMMARY OF THE INVENTION

The present inventors have found that even for a thrombosis and/or embolism patient with severe renal impairment, the use of edoxaban at a dose of 15 mg once a day can effectively prevent thrombosis and/or embolism while avoiding the risk of bleeding. The present inventors have also found that even for a thrombosis and/or embolism patient with severe renal impairment, edoxaban at a dose of 15 mg once a day can effectively prevent thrombosis and/or embolism with safety over a long period.

Specifically, the present invention relates to:
[1] a preventive and/or therapeutic agent for thrombosis and/or embolism in a thrombosis and/or embolism patient with severe renal impairment, containing edoxaban, wherein the edoxaban is administered at a dose of 15 mg once a day;
[2] the preventive and/or therapeutic agent according to [1], wherein the patient has creatinine clearance of 15 mL/min or higher and lower than 30 mL/min;
[3] the preventive and/or therapeutic agent according to [1], wherein the preventive and/or therapeutic agent is administered for at least 15 days continuously and/or intermittently;
[4] the preventive and/or therapeutic agent according to [1], wherein the thrombosis and/or embolism is venous thromboembolism or thrombosis and/or embolism attributed to atrial fibrillation;
[5] the preventive and/or therapeutic agent according to [4], wherein the venous thromboembolism is venous thromboembolism in a postoperative patient;
[6] the preventive and/or therapeutic agent according to [4], wherein the venous thromboembolism is acute venous thromboembolism;
[7] the preventive and/or therapeutic agent according to [4], wherein the thrombosis and/or embolism attributed to atrial fibrillation is cerebral infarction, systemic embolism, or stroke attributed to atrial fibrillation;
[8] a kit for preventing and/or treating thrombosis and/or embolism in a thrombosis and/or embolism patient with severe renal impairment, comprising a pharmaceutical composition containing edoxaban and an instruction to administer the edoxaban at a dose of 15 mg once a day; and
[9] a method for preventing and/or treating thrombosis and/or embolism in a thrombosis and/or embolism patient with severe renal impairment, comprising administering edoxaban at a dose of 15 mg once a day to the patient.

### DETAILED DESCRIPTION

First, terms used herein will be described.

The term “renal functional impairment” is used herein as a general term for “mild renal impairment”, “moderate renal impairment”, “severe renal impairment”, and “renal failure”.

The “mild renal impairment” used herein is defined in terms of CL_{CR} as CL_{CR} between 50 mL/min and 80 mL/min inclusive or an individual having CL_{CR} between 50 mL/min and 80 mL/min inclusive.

The “moderate renal impairment” used herein is defined in terms of CL_{CR} as CL_{CR} of 30 mL/min or higher but lower than 50 mL/min or an individual having CL_{CR} of 30 mL/min or higher but lower than 50 mL/min.

The “severe renal impairment” used herein is defined in terms of CL_{CR} as CL_{CR} lower than 30 mL/min or an individual having CL_{CR} lower than 30 mL/min.

The “renal failure” used herein refers to more severe renal impairment and is defined in terms of CL_{CR} as CL_{CR} lower than 15 mL/min or an individual having CL_{CR} lower than 15 mL/min. The “renal failure” generally refers to renal functional impairment requiring dialysis. The terms “renal failure”, “renal failure patient”, and “peritoneal dialysis patient” used herein have the same meaning with each other and may be used interchangeably.

The severity of renal functional impairment is described herein mainly using CL_{CR} values. Those skilled in the art will understand that the severity of renal functional impairment is also indicated by an estimated glomerular filtration rate (eGFR) which can be converted to CL_{CR} and vice versa.

The term “venous thromboembolism” (VTE) is used herein as a general term for deep vein thrombosis (including deep vein thrombosis in postoperative patients and acute deep vein thrombosis) and pulmonary embolism (including pulmonary embolism in postoperative patients and acute pulmonary embolism).

The term “atrial fibrillation” (AF) used herein encompasses, but is not limited to, non-valvular atrial fibrillation (NVAF).

The term “postoperative patient” used herein refers to a patient who has undergone an operation for any reason that is not particular limited. The “postoperative patient” is not limited to a patient who is hospitalized after an operation but also encompasses a patient who has been discharged from a hospital after an operation and needs to visit the hospital for the observation of signs of thrombosis and/or embolism attributed to the operation.

The term “lower limb operation” used herein refers to any operation conducted for a lower limb, and examples thereof include, but are not particularly limited to, lower limb surgery or lower limb orthopedic surgery.

The “lower limb orthopedic surgery” used herein refers to any lower limb operation conducted in the orthopedic field, and examples thereof include, but are not particularly limited to, TKR, THR, and HFS.

The term “prevention” used herein refers to the prevention of a disease and/or a pathological condition from occurring and also encompasses secondary prevention.

Creatinine clearance (CL_{CR}) is calculated herein according to the Cockcroft-Gault equation:

For male: {(140−age)×body weight (kg)}÷{72×serum creatinine level (mg/dL)}

For female: [{(140−age)×body weight (kg)}÷{72×serum creatinine level (mg/dL)}]×0.85 Cockcroft-Gault equation

Next, the present invention will be described.

N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (I):

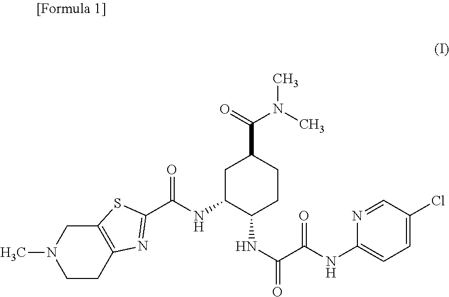 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

is called edoxaban (N-(5-chloropyridin-2-yl)-N′-[(1S,2R,4S)-4-(N,N-dimethylcarbamoyl)-2-(5-methyl-4,5,6,7-tetrahydro[1,3]thiazolo[5,4-c]pyridine-2-carboxamido)cyclohexyl]oxamide) as International Nonproprietary Name (INN).

LIXIANA (registered trademark) tablets, which comprise edoxaban tosilate monohydrate represented by the following formula (Ia):

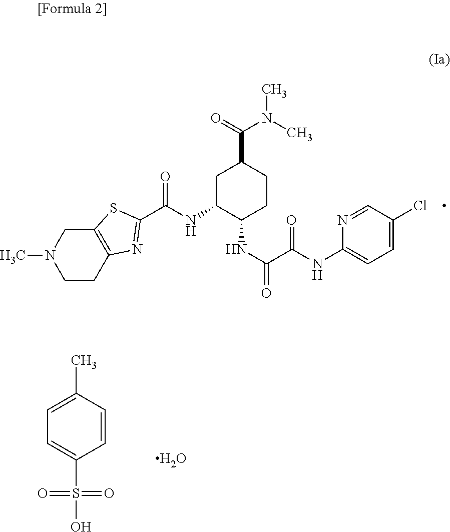 [SMILES: CN1CCc2nc(C(=O)N[C@@H]3C[C@@H](C(=O)N(C)C)CC[C@@H]3NC(=O)C(=O)Nc3ccc(Cl)cn3)sc2C1 ; Cc1ccc(S(=O)(=O)O)cc1 ; O]

as an active ingredient, are sold in Japan.

Approximately 50% of the edoxaban tosilate monohydrate absorbed in the body is excreted unchanged from the kidney, and the plasma level of edoxaban rises along with a decrease in CL_{CR}. In a clinical pharmacological trial targeting individuals with renal functional impairment, a rise in area under the plasma (blood) concentration-time curve from 0 to 24 hours after administration (referred to as AUC₀₋₂₄ₕ herein), a tendency to prolong t_{1/2}, a rise in plasma edoxaban concentration 24 hours after administration (referred to as C₂₄ₕ herein), and reduction in renal clearance (referred to as CL_{R} herein) were observed in the individuals with renal functional impairment compared with healthy adults. AUC₀₋₂₄ₕ and C₂₄ₕ of individuals with moderate renal impairment increased 1.65 times and 2.52 times respectively, when compared with those of healthy adults. Individuals with severe renal impairment exhibited prolonged t_{1/2} and a rise in C₂₄ₕ compared with the individuals with moderate renal impairment, though no large difference was observed in AUC₀₋₂₄ₕ or the maximum plasma concentration (referred to as Cₘₐₓ herein) therebetween. The degree of the rise in C₂₄ₕ in the individuals with severe renal impairment was approximately 3 times that in healthy adults and approximately 2 times that in the individuals with mild renal impairment (Table 1).

TABLE 1
Pharmacokinetic parameters following single-dose administration of Edoxaban
at a dose of 15 mg to individuals with renal functional impairment

| | | Subject population^{a)} | | | | |
|---|---|---|---|---|---|---|
| | | Healthy | Mild renal | Moderate renal | Severe renal | Peritoneal |
| | | adult | impairment | impairment | impairment | dialysis patient |
| | | The number of test subjects evaluated | | | | |
| | | 8 | 8 | 8 | 8 | 8 |
| AUC_{0-24 h} | Geometric least | 440 | 569 | 726 | 661 | 770 |
| (ng · h/mL) | squares mean | | | | | |
| | Geometric least | — | 1.29 | 1.65 | 1.50 | 1.75 |
| | squares mean ratio to | | (1.06, 1.58) | (1.35, 2.01) | (1.22, 1.84) | (1.42, 2.16) |
| | healthy adult(90% CI) | | | | | |
| Cₘₐₓ | Geometric least | 88.8 | 104 | 113 | 83.6 | 83.8 |
| (ng/mL) | squares mean | | | | | |
| | Geometric least | — | 1.17 | 1.27 | 0.941 | 0.944 |
| | squares mean ratio to | | (0.850, 1.60) | (0.930, 1.74) | (0.682, 1.30) | (0.679, 1.31) |
| | healthy adult(90% CI) | | | | | |
| tₘₐₓ (h) | Least squares mean | 1.50 | 1.88 | 1.38 | 1.70 | 2.37 |
| | Least squares mean | — | 0.375 | −0.125 | 0.200 | 0.875 |
| | difference from | | (−0.409, 1.16) | (−0.909, 0.659) | (−0.584, 0.984) | (0.090, 1.66) |
| | healthy adult(90% CI) | | | | | |
| t_{1/2} (h) | Geometric least | 8.60 | 8.15 | 9.44 | 16.9 | 12.2 |
| | squares mean | | | | | |
| | Geometric least | — | −0.440 | 0.847 | 8.33 | 3.65 |
| | squares mean ratio to | | (−5.26, 4.37) | (−3.97, 5.66) | (3.51, 13.1) | (−1.17, 8.46) |
| | healthy adult(90% CI) | | | | | |
| C_{24 h} | Geometric mean | 2.34 | 3.44 | 5.90 | 6.88 | 8.24 |
| (ng/mL) | CV % | 28.1 | 62.5 | 38.4 | 36.2 | 53.9 |
| CL_{R} | Geometric mean | 197 | 121 | 67.4 | 32.5 | — |
| (mL/min) | CV % | 16.5 | 37.8 | 37.8 | 49.3 | — |

^{a)}Healthy adult CL_{CR} > 80 mL/min, Mild renal impairment 50 mL/min ≦ CL_{CR} ≦ 80 mL/min, Moderate renal impairment 30 mL/min ≦ CL_{CR} < 50 mL/min, Severe renal impairment CL_{CR} < 30 mL/min (nondialyzed individual)

Assessment Reports, etc. for the LIXIANA (registered trademark) tablet describe that the applicant contemplated that the drug could be administered at a dose of 15 mg daily for patients having CL_{CR} lower than 30 mL/min, on the basis of analysis results of Japan TKR phase-II trial targeting the prevention of VTE in patients undergoing TKR, THR, or HFS. The Assessment Reports, etc. also describe the judgment by Pharmaceuticals and Medical Devices Agency and by the expert committee of Expert Council that the use of the LIXIANA (registered trademark) tablet should be contraindicated to patients having CL_{CR} lower than 30 mL/min. The reasons for the judgment include: the unknown safety of the LIXIANA (registered trademark) tablet for the patients having CL_{CR} lower than 30 mL/min; the benefit of avoiding VTE may be accompanied by the unacceptable risk of bleeding; and the risk of VTE can be reduced by an approach other than the administration of the anticoagulant drug (Non-patent literature 5 to 9).

The package insert of the LIXIANA (registered trademark) tablet describes: the LIXIANA (registered trademark) tablet should be used for a patient undergoing lower limb orthopedic surgery only during hospitalization after the operation as a rule; the administration period should be determined in consideration of the risks of venous thromboembolism and bleeding in each individual patient; not to continuously administer the drug thoughtlessly after the risk of venous thromboembolism is reduced; and the absence of clinical studies to evaluate the efficacy and safety of 15-day or longer administration of edoxaban in patients undergoing lower limb orthopedic surgery in Japan.

When a FXa inhibitor is used as an anticoagulant agent in the prevention and/or treatment of thrombosis and/or embolism in a postoperative patient, its administration period is short, for example, for: 5 days after the operation (e.g., after surgery and/or orthopedic surgery; the same holds true for the description below), 1 week after the operation, 10 days after the operation, or 2 weeks after the operation. Alternatively, when the FXa inhibitor is used as an anticoagulant agent in the prevention and/or treatment of acute thrombosis and/or embolism or in the prevention and/or treatment of thrombosis and/or embolism in an AF patient, its administration period is long, for example, for: 5 days or longer, 1 week or longer, 10 days or longer, 2 weeks or longer, 15 days or longer, 1 month or longer, 2 months or longer, 3 months or longer, 4 months or longer, 5 months or longer, or half a year or longer, or 1 year or longer.

Both AF and chronic kidney disease (referred to as CKD herein) are diseases whose prevalence increases with aging. CKD patients have been reported to have particularly high AF prevalence. And, it is said that reduced renal function in AF patients is an independent factor increasing the risk of occurrence of thromboembolism. For those reason, AF patients with severe renal impairment are a population at a high risk of ischemic stroke or systemic thromboembolism in need of long-term anticoagulant therapy. Meanwhile, patients with severe renal impairment exhibit reduced platelet functions and are thus also a population at a high risk of bleeding. At the moment, only warfarin is used as an anticoagulant drug for AF patients with severe renal impairment in Japan and foreign medical practice. Unfortunately, it has been reported that: the dose of warfarin is difficult to adjust due to the difference in the manifestation of pharmacological effect among individuals and the interaction with foods or drugs; and the administration of warfarin tends to increase the incidence of major bleeding depending on the severity of renal functional impairment. Thus, it is important to develop a more manageable, orally administrable anticoagulant drug that is excellent in the balance between the risk of bleeding and preventive effect on the occurrence of thromboembolism in the AF patient with severe renal impairment.

No statistical evaluation has been made yet on the safety and/or efficacy of edoxaban administered over a long period (e.g., 15 days or longer) to a patient population in need of treatment of thrombosis and/or embolism with severe renal impairment.

The feature of the pharmaceutical composition or the preventive and/or therapeutic agent of the present invention containing edoxaban is that it is administered for at least 5 days consecutively and/or intermittently. Preferably, the pharmaceutical composition or the preventive and/or therapeutic agent of the present invention containing edoxaban is administered for: 5 days or longer, 1 week or longer, 10 days or longer, 2 weeks or longer, 15 days or longer, 1 month or longer, 2 months or longer, 3 months or longer, 4 months or longer, 5 months or longer, half a year or longer, or 1 year or longer consecutively and/or intermittently. For use in the reduction of occurrence of venous thromboembolism in a postoperative patient, the pharmaceutical composition or the preventive and/or therapeutic agent of the present invention containing edoxaban is preferably administered for 1 to 14 days consecutively and/or intermittently. For use in the prevention and/or treatment of acute venous thromboembolism, the pharmaceutical composition or the preventive and/or therapeutic agent of the present invention containing edoxaban is preferably administered for: 5 days or longer, 1 week or longer, 10 days or longer, 2 weeks or longer, 15 days or longer, 1 month or longer, 2 months or longer, 3 months or longer, 4 months or longer, 5 months or longer, half a year or longer, or 1 year or longer consecutively and/or intermittently. For use in the prevention and/or treatment of thrombosis and/or embolism attributed to atrial fibrillation, for example, cerebral infarction, systemic embolism, or stroke attributed to atrial fibrillation, the pharmaceutical composition or the preventive and/or therapeutic agent of the present invention containing edoxaban is preferably administered for: 5 days or longer, 1 week or longer, 10 days or longer, 2 weeks or longer, 15 days or longer, 1 month or longer, 2 months or longer, 3 months or longer, 4 months or longer, 5 months or longer, half a year or longer, or 1 year or longer consecutively and/or intermittently.

The “severe renal impairment” used herein refers to CL_{CR} lower than 30 mL/min or an individual having such a CL_{CR} value. In various examples, the range of CL_{CR} in severe renal impairment includes 15 mL/min or higher but lower than 30 mL/min.

The “postoperative patient” used herein preferably refers to a patient 14 days or earlier after an operation.

Examples of the postoperative patient to which the pharmaceutical composition or the preventive and/or therapeutic agent of the present invention is applied preferably include patients who have undergone a lower limb operation, more preferably patients who have undergone a lower limb operation conducted in the orthopedic field, even more preferably patients who have undergone TKR, THR, or HFS.

The dosage form of the pharmaceutical composition or the preventive and/or therapeutic agent of the present invention can be any orally administrable dosage form and may be a solid or nonsolid preparation, with a solid preparation preferred.

The orally administrable solid preparation is not particularly limited and is preferably tablets (including orally disintegrating tablets), granules (including fine granules), powders, or capsules. The orally administrable solid preparation can be produced by adopting a well-known method for producing solid preparations.

When the pharmaceutical composition of the present invention is a solid preparation, this solid preparation may comprise a coating agent. The coated solid preparation is not limited to coated solid preparations such as coated tablets and also encompasses various solid preparations comprising coating agents. For example, a solid preparation containing edoxaban or a pharmacologically acceptable salt thereof, or a solvate thereof, wherein coating agents are formulated in a matrix form in the solid preparation is also included in the present invention.

The present invention also relates to a kit for preventing and/or treating thrombosis and/or embolism in a thrombosis and/or embolism patient with severe renal impairment, comprising a pharmaceutical composition containing edoxaban and an instruction to administer the edoxaban at a dose of 15 mg once a day. The pharmaceutical composition contained in the kit can have any orally administrable dosage form as described above. Examples of the form of the kit include, but are not particularly limited to, packaged containers comprising a packaged pharmaceutical composition containing edoxaban and an instruction (e.g., package insert) that guides how to take the drug. The instruction may be present as an independent sheet such as a package insert or may be attached to the container containing the pharmaceutical composition containing edoxaban. The accompanying method thereof is not particularly limited.

The present invention further relates to a method for preventing and/or treating thrombosis and/or embolism in a thrombosis and/or embolism patient with severe renal impairment, comprising administering 15 mg of edoxaban to the patient once a day. The edoxaban can be administered at a dose of 15 mg to the patient once a day through any route that is not particularly limited and, preferably, is orally administered. For the oral administration, both solid and non-solid preparations can be used, and, preferably a solid preparation, more preferably a tablet is administered. The solid preparation of the present invention can be in any form by which 15 mg of edoxaban is administered once a day. 15 mg of edoxaban may be contained in one preparation (e.g., in one tablet or one packet) or may be divided into a plurality of preparations (e.g., two or more tablets or two or more packets). Alternatively, the solid preparation of the present invention is used by splitting. In this form, one dose after the splitting may contain 15 mg of edoxaban. When the solid preparation of the present invention is a tablet, the tablet may be a tablet to be used by splitting which is designed so that one dose after the splitting contains 15 mg of edoxaban (in this context, the tablet is designed so that its efficacy and safety are appropriately secured for the use of each portion of the split tablet) or may be a tablet containing, for example, 30 mg of edoxaban to be divided into two portions in use (in this context, the tablet is designed so that the efficacy and safety of one dose after the splitting are appropriately secured). Preferable examples of the tablet of the present invention include tablets each containing 15 mg of edoxaban.

Next, the present invention will be described in detail with reference to Examples. However, the present invention is not intended to be limited to them by any means.

### EXAMPLES Example 1

### Late Phase-II Trial Targeting NVAF Patient in Japan

In Japan, edoxaban 30 mg×1/day, edoxaban 45 mg×1/day, or edoxaban 60 mg×1/day, or warfarin (whose PT-INR was adjusted to 2.0 to 3.0 (1.6 to 2.6 for 70 years or older)) was orally administered for 12 weeks to each of 519 NVAF patients to evaluate the incidence of thromboembolic events and the incidence of bleeding events.

The only thromboembolic event was one cerebral infarction that occurred in one subject in the edoxaban 45 mg group.

Major bleeding occurred in 3 subjects (2.2%) in the edoxaban 45 mg group and 2 subjects (1.5%) in the edoxaban 60 mg group. The incidence of major bleeding or the incidence of major bleeding or clinically relevant non-major bleeding was not statistically significantly different between the warfarin group and each edoxaban group. Likewise, no significant difference was seen in the paired comparison among the edoxaban groups. In addition, a statistically significant dose-response relationship was not observed.

The incidence of bleeding events (major bleeding, clinically relevant non-major bleeding, and minor bleeding in total) was 18.5% (24/130) in the edoxaban 30 mg group, 22.4% (30/134) in the edoxaban 45 mg group, 27.7% (36/130) in the edoxaban 60 mg group, and 20.0% (25/125) in the warfarin group, demonstrating a rise in incidence along with increase in edoxaban dose. The edoxaban 60 mg group exhibited a slightly higher incidence than that of the warfarin group. No statistically significant difference, however, was seen between the warfarin group and each edoxaban group. Likewise, no significant difference was seen in the paired comparison among the edoxaban groups. In addition, statistically significant dose-response relationship was not observed.

A population pharmacokinetic analysis was performed using the plasma edoxaban concentration data of a Japanese late phase-II study in patients with NVAF. As a result, CL_{CR} for clearance was selected as a covariate significantly influencing the pharmacokinetics of edoxaban.

Meanwhile, the relationship of renal functions with bleeding, which was the side effect based on the anticoagulant effect of edoxaban, was not clearly confirmed particularly due to a small number of subjects with moderate renal impairment (CL_{CR}: 30 mL/min or higher but lower than 50 mL/min) and few subjects with severe renal impairment (CL_{CR}: lower than 30 mL/min) (Table 2).

TABLE 2
Incidence of bleeding events by CL_{CR} in late
phase-II trial targeting NVAF patient in Japan

| | Major bleeding | | | | | |
|---|---|---|---|---|---|---|
| | or clinically relevant | | | | | |
| | non-major bleeding | | | Bleeding event^{a)} | | |
| | 30 mg | 45 mg | 60 mg | 30 mg | 45 mg | 60 mg |
| CL_{CR} < 30 | — | — | 0% | — | — | 100.0% |
| (mL/min) | | | (0/1) | | | (1/1) |
| 30 ≦ CL_{CR} < | 6.7% | 9.5% | 0% | 20.0% | 28.6% | 26.7% |
| 50 (mL/min) | (1/15) | (2/21) | (0/15) | (3/15) | (6/21) | (4/15) |
| 50 ≦ CL_{CR} ≦ | 1.4% | 4.2% | 10.3% | 20.3% | 26.4% | 30.9% |
| 80 (mL/min) | (1/69) | (3/72) | (7/68) | (14/69) | (19/72) | (21/68) |
| 80 < CL_{CR} | 0% | 4.9% | 0% | 15.2% | 12.2% | 21.7% |
| (mL/min) | (0/46) | (2/41) | (0/46) | (7/46) | (5/41) | (10/46) |

^{a)}major bleeding, clinically relevant non-major bleeding, and minor bleeding in total

The relationship between bleeding events and pharmacokinetic parameters was studied using logistic regression models. As a result, the incidence of bleeding events (major bleeding, clinically relevant non-major bleeding, and minor bleeding in total) was confirmed to correlate with AUC₀₋₂₄ₕ or Cₘₐₓ at steady state or with the plasma edoxaban concentration at trough (Cₘᵢₙ).

### Example 2

### Phase-III Trial Targeting NVAF Patient with Severe Renal Impairment

### <Clinical Trial Protocol>

After obtaining consent from the subject for participation in the study, his/her CL_{CR} values were calculated using to the Cockcroft-Gault equation at screening to evaluate the renal function of the subject to confirm that he/she was a NVAF patient with severe renal impairment (SRI) (CL_{CR}: 15 mL/min or higher but lower than 30 mL/min (except for hemodialysis patients)) or with normal renal functions or mild renal impairment (MiRI) (CL_{CR},: 50 mL/min or higher). The subjects were enrolled to the study after further examination to confirm he/she fulfilled the inclusion/exclusion criteria.

For male: {(140−age)×body weight (kg)}÷(72×serum creatinine level (mg/dL)}

For female: [({140−age)×body weight (kg)}]÷(72×serum creatinine level (mg/dL))]×0.85 Cockcroft-Gault equation

Subjects with SRI and NVAF received 15 mg of edoxaban once a day for 12 weeks. Subjects with normal renal functions and NVAF or subjects with MiRI and NVAF received 30 mg or 60 mg of edoxaban once a day for 12 weeks. The safety and pharmacokinetics of edoxaban in subjects with SRI and NVAF were compared with those in subjects with normal renal functions and NVAF or subjects with MiRI and NVAF.

### Selection of Study Subject:

Inclusion criteria are shown below.
1) NVAF patient with SRI (CL_{CR}: 15 mL/min or higher but lower than 30 mL/min) or with normal renal functions or MiRI (CL_{CR}: 50 mL/min or higher)
2) Aged 20 and over
3) Individual who has been confirmed to have AF by electric recording within the past 12 months, can be adapted for anticoagulant therapy, and is to be subjected to anticoagulant therapy during the study period
4) Individual having at least one risk factor for thromboembolism

### Safety Endpoint:

1) Incidence of major bleeding or clinically relevant non-major bleeding
2) Incidence of bleeding events (major bleeding, clinically relevant non-major bleeding, or minor bleeding)
3) Incidence of major bleeding
4) Incidence of clinically relevant non-major bleeding
5) Incidence of adverse events
6) Incidence of adverse drug reaction

### Efficacy Evaluation:

Cerebral infarction and systemic embolism are defined as thromboembolic events, and the presence or absence of occurrence thereof is examined from the start point of administration of the investigational new drug to the point of visiting a hospital at a follow-up stage (after the completion of a treatment period or on the 2nd week after discontinuation of treatment).

### Efficacy Results:

No cerebral infarction or systemic embolism occurred in any of the treatment groups.

### Safety Results:

No notable difference was found in the incidence of any adjudicated bleeding events between the SRI 15-mg group and the Normal/MiRI low-dose or high-dose group.

The incidence of any adjudicated bleeding events was 20.0% (10 of 50 subjects) in the SRI 15-mg group, 22.7% (5 of 22) in the Normal/MiRI low-dose group, and 23.8% (5 of 21) in the Normal/MiRI high-dose group (see Table below). No adjudicated major bleeding events occurred in any of the treatment groups. The incidence of adjudicated clinically relevant non-major bleeding events was 0% (0 of 50) in the SRI 15-mg group, 0% (0 of 22) in the Normal/MiRI low-dose group, and 4.8% (1 of 21) in the Normal/MiRI high-dose group. Most adjudicated bleeding events were minor bleeding events in all treatment groups. No notable difference was found in the type of common adjudicated bleeding events between the SRI 15-mg group and the Normal/MiRI low-dose or high-dose group.

TABLE 3
Incidence of Adjudicated Bleeding Events (Safety Analysis Set)

| | SRI | Normal/MiRI | |
|---|---|---|---|
| | edoxaban | edoxaban | edoxaban |
| | 15 mg | Low-dose | High-dose |
| Number of subjects | 50 | 22 | 21 |
| Major bleeding events | 0 (0.0) | 0 (0.0) | 0 (0.0) |
| Clinically relevant non-major | 0 (0.0) | 0 (0.0) | 1 (4.8) |
| bleeding events | | | |
| Any bleeding events | 10 (20.0) | 5 (22.7) | 5 (23.8) |

Number of subjects with events (%)

No notable differences were found in the incidence and type of adverse events between the treatment groups. Most of the adverse events were related to bleeding or infection. Moderate or severe adverse events occurred in the SRI 15-mg group; no such events occurred in the Normal/MiRI low-dose or high-dose group. No deaths were reported in any of the treatment groups. All of the serious adverse events were reported in the SRI 15-mg group. All of the serious adverse events were considered unrelated to the study drug.

Adverse events leading to study drug discontinuation occurred most frequently in the SRI 15-mg group. However, no serious adverse events or adverse events leading to study drug discontinuation affected the known safety profile of edoxaban.

Differences in some laboratory parameters, including red blood cell count, hemoglobin, hematocrit, albumin, BUN, and serum creatinine, were found between the SRI 15-mg group and the Normal/MiRI low-dose or high-dose group. However, no notable difference was found in the time course of laboratory values after initiation of study treatment, in shifts in laboratory values, or in changes in vital signs over time among the treatment groups.

### Conclusions:

This study demonstrated that administration of 15 mg of edoxaban for 12 weeks in patients with SRI did not result in a marked increase in bleeding compared to the low dose or high dose of edoxaban in patients with Normal/MiRI.

1. A method for treating thrombosis and/or embolism in a patient with non-valvular atrial fibrillation and severe renal impairment, comprising administering a therapeutic agent containing 15 mg of edoxaban once a day.

2. The method according to claim 1, wherein the patient has creatinine clearance of 15 mL/min or higher but lower than 30 mL/min.

3. The method according to claim 1, wherein the therapeutic agent is administered for at least 15 days continuously and/or intermittently.

4. The method according to claim 1, wherein the thrombosis and/or embolism is venous thromboembolism or thrombosis and/or embolism attributed to atrial fibrillation.

5. The method according to claim 4, wherein the venous thromboembolism is acute venous thromboembolism.

6. The method according to claim 4, wherein the thrombosis and/or embolism attributed to atrial fibrillation is cerebral infarction, systemic embolism, or stroke attributed to atrial fibrillation.

7. (canceled)

8. (canceled)
